(19) United States
(12) Patent Application Publication
(10) Pub. No.: US 2015/0320764 A1
(43) Pub. Date: Nov. 12, 2015

(54) TREATING FEMALE SEXUAL AROUSAL DISORDER AND RELATED SYMPTOMS

(71) Applicant: NexMed Holdings, Inc., San Diego, CA (US)

(72) Inventors: Susan Meier-Davis, San Diego, CA (US); Daniel W. Frank, Broomall, PA (US); Richard Martin, San Diego, CA (US)

(73) Assignee: NEXMED HOLDINGS, INC., San Diego, CA (US)

(21) Appl. No.: 14/707,984

(22) Filed: May 8, 2015

Related U.S. Application Data
(60) Provisional application No. 61/991,157, filed on May 9, 2014.

(51) Int. Cl. A61K 31/5575; A61K 9/00; A61K 31/221

(52) U.S. Cl. CPC A61K 31/5575; A61K 31/221; A61K 9/0014

(57) ABSTRACT

This invention relates to treating female sexual arousal disorder and/or improving female sexual response, and related symptoms, preferably with topical prostaglandin compositions.

### CROSS REFERENCE TO RELATED APPLICATION

This application claims priority to U.S. provisional application No. 61/991,157, filed May 9, 2014, the content of which is incorporated herein by reference in its entirety.

### FIELD OF INVENTION

This invention relates to treating female sexual arousal disorder and/or improving female sexual response, and related symptoms, preferably with topical prostaglandin compositions.

### STATE OF THE ART

U.S. Pat. Nos. 6,825,234 and 6,486,207 disclose topical administration of prostaglandin related to female sexual dysfunction. However, no correlation is provided between improving female sexual arousal disorder and/or female sexual response on one hand and the temperature associated in female genitalia, on the other.

### SUMMARY

Ten healthy premenopausal women were topically administered Femprox® and an over-the counter (OTC) lubricant to their clitoris and anterior vaginal wall in a cross-over study design. Continuous temperature monitoring of the vestibule, clitoris and vulva was conducted for one hour in women watching a non-sexual, i.e., travel film. With each application, subjects completed questionnaires assessing their genital sensations relative to feelings, maximum intensity and duration of effect and adverse events recorded.

With each application, Femprox® induced an increase whilst the OTC lubricant resulted in decreased temperature relative to baseline measurements in all subjects. Femprox® application induced a statistically significant increase in temperature of the vestibule, clitoris and vulva relative to the OTC lubricant. Sustained treatment differences occurred at 11 minutes post application for the vestibule, 19 minutes for the clitoris and 9 minutes for the vulva. The temperature differences were maintained for the duration of the one-hour assessment period.

Nine of the ten women reported genital sensations with the Femprox® application indicating a neutral to positive response in eight women and one negative response. The maximal intensity described by the nine women after the Femprox® application ranged from immediate to greater than 30 minutes. The duration of effect spanned from 5 minutes to more than thirty minutes in seven women with two women not able to determine the duration. In contrast, three of ten women administered the OTC lubricant had genital sensations and described a neutral response, each describing a different time to maximum intensity and duration of effect. No adverse events were reported.

Topical administration of Femprox® to healthy premenopausal women induced increases in genital temperatures of the vestibule, clitoris and vulva within 20 minutes relative to OTC lubricant that was maintained for the one-hour duration. The majority of women administered Femprox® reported a neutral to positive response with a maximum intensity within ten minutes lasting longer than 30 minutes. Although a limited number of women were tested, a trend for concordance between the physiological and subjective assessments was recognized.

In one aspect, this invention provides a method of enhancing female sexual response during a sexual engagement, comprising topically administering a composition to the patient's genitalia wherein said composition is configured to increase the temperature of one or more of the vestibule, clitoris and/or vulva.

In one embodiment, the composition comprises an effective amount of alprostadil and an effective amount of dodecyl 2-(N,N-dimethylamino)propionate (DDAIP) and associated metabolites.

In another aspect, this invention provides a composition comprising an effective amount of alprostadil and an effective amount of dodecyl 2-(N,N-dimethylamino)propionate (DDAIP) and associated metabolites for use in increasing the temperature of one or more of the vestibule, clitoris and/or vulva topically administering a composition to the patient's genitalia prior to sexual engagement to enhance female sexual response.

In one aspect, this invention provides a method of increasing vestibule temperature in a female patient in need thereof, the method comprising topically administering a composition comprising alprostadil and dodecyl 2-(N,N-dimethylamino)propionate (DDAIP) to the patient's genitalia.

In another aspect, this invention provides a method of increasing clitoral temperature in a female patient in need thereof, the method comprising topically administering a composition comprising alprostadil and DDAIP to the patient's genitalia.

In another aspect, this invention provides a method of increasing vulvar temperature in a female patient in need thereof, the method comprising topically administering a composition comprising alprostadil and DDAIP to the patient's genitalia.

In another aspect, this invention provides a method of enhancing female sexual response during a sexual engagement, comprising topically administering a composition comprising alprostadil and DDAIP to the genitalia of a female in need thereof between about 10 minutes to about 90, or about 20 to about 60 minutes prior to the sexual engagement by the female, wherein one or more of vestibule, clitoral or vulvar temperature of the female is increased.

In another aspect, this invention provides a method of enhancing female sexual response during a sexual engagement, comprising topically administering a composition comprising alprostadil and DDAIP to the genitalia of a female in need thereof between about 10 minutes to about 90, or about 20 to about 60 minutes prior to the sexual engagement by the female, wherein the enhanced female sexual response is experienced within, about 10, about 15, about 20 minutes, or about 30 minutes over a baseline.

In another aspect, this invention provides a composition comprising an effective amount of alprostadil and an effective amount of dodecyl 2-(N,N-dimethylamino)propionate (DDAIP) for use in enhancing female sexual response during a sexual engagement by topical administration to the genitalia of a female in need thereof between about 10 minutes to 90 minutes prior to said sexual engagement.

In another aspect, this invention provides the use of a composition comprising an effective amount of alprostadil and an effective amount of dodecyl 2-(N,N-dimethylamino)propionate (DDAIP), for the manufacture of a medicament for use in enhancing female sexual response during a sexual engagement by topical administration to the genitalia of a female in need thereof between about 10 minutes to 90 minutes prior to said sexual engagement.

In another aspect, this invention provides a method of enhancing female sexual response during a sexual engagement, comprising identifying a female in need thereof during a 120 minute window of time and topically administering a composition comprising alprostadil and DDAIP to one or more of vestibule, clitoral or vulvar region of the female's genitalia between about 10 minutes prior to and up until about 100 minutes into said 120 minute window. In one embodiment, the identified female experiences enhanced female sexual response within, about 10, about 15, about 20 minutes, or about 30 minutes over a baseline. In one embodiment, the identified female undergoes a temperature increase, preferably a substantial temperature increase, such as those illustrated in FIG. 1, 2, or 3, within, about 10, about 15, about 20 minutes, or about 30 minutes over a baseline.

As used herein, a baseline can be measured based on the temperature increase and duration of increase in a female applied a non-steroidal, placebo, such as a lubricant.

In another aspect, this invention provides a composition comprising an effective amount of alprostadil and an effective amount of dodecyl 2-(N,N-dimethylamino)propionate (DDAIP) for use in enhancing female sexual response during a sexual engagement within a 120 minute window of time by topical administration to one or more of vestibule, clitoral or vulvar region of the female's genitalia between about 10 minutes prior to and up until about 100 minutes into said 120 minute window of time.

In another aspect, this invention provides the use of a composition comprising an effective amount of alprostadil and an effective amount of dodecyl 2-(N,N-dimethylamino)propionate (DDAIP) for the manufacture of a medicament for use in enhancing female sexual response during a sexual engagement within a 120 minute window of time by topical administration to one or more of vestibule, clitoral or vulvar region of the female's genitalia between about 10 minutes prior to and up until about 100 minutes into said 120 minute window of time.

In one embodiment, an effective temperature increase is observed within about 5-about 20, or about 10 minutes. As used herein, an effective temperature increase is the temperature increase that is desired for beneficial female sexual arousal or female sexual response. In another embodiment, the effective temperature is maintained for about 45 minutes to about 2 hours, or about 1-about 1.5 hours. In another embodiment, the composition is a cream. In another embodiment, the composition comprises about 0.025-about 10% weight percent, or about 0.1-about 5% weight percent of alprostadil. In another embodiment, the composition comprises 0.42% weight percent of alprostadil. In another embodiment, the composition comprises 0.0.025-40% weight percent of DDAIP. In another embodiment, the composition comprises 0.5-35% weight percent of DDAIP.

In one embodiment, the vestibule temperature increase is at least 0.5% for a 90 minute duration. In one embodiment, the vulvar temperature increase is at least 0.7% for a 90 minute duration. In one embodiment, the clitoral temperature increase is at least 0.8% for a 90 minute duration.

In one embodiment, the composition is stable at room temperature. In some embodiments, it is stable for up to a week, 2 weeks, a month, 2 months, 6 months, or more. In another embodiment, the composition is a non-aqueous composition. In another embodiment, the composition is free of substantially free of a C₁-C₃ alcohol, such as ethyl alcohol. Water and or alcohol can be added as a diluent to the composition prior to patient application to form a final combined composition.

### BRIEF DESCRIPTION OF THE DRAWINGS

FIG. 1 graphically illustrates mean change from baseline for vestibule temperature for Femprox® (FEM) and placebo (OTC lubricant, PBO) administered patients.

FIG. 2 graphically illustrates mean change from baseline for clitoral temperature for FEM and PBO administered patients.

FIG. 3 graphically illustrates mean change from baseline for vulvar temperature for FEM and PBO administered patients.

### DETAILED DESCRIPTION

### Definitions

The singular forms “a,” “an,” and “the” and the like include plural referents unless the context clearly dictates otherwise. Thus, for example, reference to “a compound” includes both a single compound and a plurality of different compounds.

The term “about” when used before a numerical designation, e.g., temperature, time, amount, and concentration, including a range, indicates approximations which may vary by .+−10%, +−5% or +−1%.

“Administration” refers to introducing an agent into a patient. A therapeutic amount can be administered, which can be determined by the treating physician or the like. The related terms and phrases “administering” and “administration of”, when used in connection with a compound or pharmaceutical composition (and grammatical equivalents) refer both to direct administration, which may be administration to a patient by a medical professional or by self-administration by the patient, and/or to indirect administration, which may be the act of prescribing a drug. For example, a physician who instructs a patient to self-administer a drug and/or provides a patient with a prescription for a drug is administering the drug to the patient. In any event, administration entails delivery to the patient of the drug.

“Comprising” shall mean that the methods and compositions include the recited elements, but not exclude others. “Consisting essentially of” when used to define methods and compositions, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transitional terms and phrases are within the scope of this invention.

“Pharmaceutically acceptable” refers to non-toxic material suitable for in vivo and preferably human administration.

“Effective amount” refers to an amount of a drug or an agent that when administered to a patient suffering from a condition, will have the intended therapeutic effect, e.g., alleviation, amelioration, palliation or elimination of one or more manifestations of the condition in the patient. The effective amount will vary depending upon the subject and the condition being treated, the weight and age of the subject, the severity of the condition, the particular composition or excipient chosen, the dosing regimen to be followed, timing of administration, the manner of administration and the like, all of which can be determined readily by one of ordinary skill in the art. The full therapeutic effect does not necessarily occur by administration of one dose, and may occur only after administration of a series of doses. Thus, an effective amount may be administered in one or more administrations.

“Patient or subject” refers to a human female.

“PG” as used in PGE₁ or PGA₁ refers to prostaglandin.

“Treatment”, “treating”, and “treat” are defined as acting upon a disease, disorder, or condition with an agent to reduce or ameliorate the harmful or any other undesired effects of the disease, disorder, or condition and/or its symptoms and produce beneficial or desired clinical results. Treatment, as used herein, covers the treatment of a female human patient, and includes: (a) reducing the risk of occurrence of the condition in a patient determined to be predisposed to the disease but not yet diagnosed as having the condition, (b) impeding the development of the condition, and/or (c) relieving the condition, i.e., causing regression of the condition and/or relieving one or more symptoms of the condition.

### DESCRIPTIVE EMBODIMENTS

In one embodiment, alprostadil includes pharmaceutically acceptable carboxyl esters of alprostadil, pharmaceutically acceptable salts of each thereof, and pharmaceutically acceptable solvates of each of the above, and metabolites of each of the foregoing.

Generally, the prostaglandin, such as alprostadil, is present in the composition in an amount of about 0.07 to about 1 percent, preferably about 0.1 to about 1 percent by weight based on the total weight of the composition. In one preferred embodiment, prostaglandin E₁ is present in an amount of about 0.07 to about 0.4 percent by weight based on the total weight of the composition. The prostaglandin can be dissolved or substantially uniformly dispersed in the topical composition. It is preferably soluble (and dissolved) in the topical composition.

In other embodiments, alprostadil or its salts, esters, and solavates as utilized herein can be replaced completely or partially with PGA₁, PGB₁, PGF_{1α}, 19-hydroxy-PGA₁, 19-hydroxy-PGB₁, PGE₂, PGA₂, PGB₂, 19-hydroxy-PGA₂, 19-hydroxy-PGB₂, PGE₃, PGF_{3α} as well as semisynthetic or synthetic derivatives of natural prostaglandins, including carboprost tromethamine, dinoprost tromethamine, dinoprostone, lipoprost, gemeprost, metenoprost, sulprostone and tiaprost. Alprostadil and the other prostaglandins disclosed in this paragraph and those others well known to the skilled artisan, are referred to as “Prostaglandin E (PGE) group compounds.”

In some embodiments, the DDAIP utilized herein includes metabolites of DDAIP. In some embodiments, the DDAIP utilized herein can be replaced partly or completely with another penetration enhancer that is an N,N-di(C₁-C₈)alkylamino substituted, (C₄-C₁₈)alkyl(C₂-C₁₈)carboxylic esters or pharmaceutically acceptable acid addition salts thereof. As used herein, the term “(C₄-C₁₈)alkyl(C₂-C₁₈)carboxylic ester” means an ester of a (C₄-C₁₈alcohol and a (C₂-C₁₈)carboxylic acid. The term “N,N-di(C₁-C₈)alkylamino substituted,” in reference to a (C₄-C₁ₛ)alkyl(C₂-C₁₈)carboxylic ester means that either the alcohol portion or the carboxylic acid portion from which the ester is prepared bears an amino substituent NRₓR_{y}, wherein Rₓ and R_{y} are each independently a (C₁-C₈)alkyl group. Preferably Rₓ and R_{y} are both methyl groups.

In one embodiment, the another penetration enhancer is of Formula I:

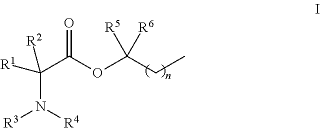

where n is an integer from 6-12 inclusive;

R¹, and R², are the same or different and are selected independently from the group consisting of H, substituted or unsubstituted, straight or branched chain C₁-C₁₀ alkyl, preferably CH₃, CHOHCH₃, CH(CH₃)₂, CH₂C₆H₆, and CH₂CH(CH₃)₂;

R³, and R⁴, are the same or different and are selected independently from the group consisting of H, substituted or unsubstituted, straight or branched chain C₁-C₁₀ alkyl, preferably CH₃, CH(CH₃)₂, CH₂CHOH, CH₂CH(CH₃)₂, and (CH₂)₂N(CH₂CH₃)₂, C₆-C₁₀ aryl or C₆-C₁₀ heteroaryl;

R⁵, and R⁶, are the same or different and are selected independently from the group consisting of H and CH₃, and pharmaceutically acceptable salts thereof.

Preferred N,N-di(C₁-C₈)alkylamino substituted, (C₄-C₁₈)alkyl(C₂-C₁₈)carboxylic esters are dodecyl-2-(N,N-dimethylamino)-propionate (DDAIP); dodecyl-2-(N,N-dimethylamino)-acetate (DDAA); 1-(N,N-dimethylamino)-2-propyl dodecanoate (DAIPD); 1-(N,N-dimethylamino)-2-propyl myristate (DAIPM); 1-(N,N-dimethylamino)-2-propyl oleate (DAIPO); and pharmaceutically acceptable acid addition salts thereof. The preparation of DDAIP and crystalline acid addition salts thereof is described in U.S. Pat. No. 6,118,020 to Büyüktimkin, et al., which is incorporated herein by reference.

In some embodiments, the composition utilized herein comprises one or more pharmaceutically acceptable excipient. Non-limiting examples of such excipients include: ethyl laurate, alkali, an acid, and a C₁-C₃ alcohol, such as ethanol. In one embodiment, the prostaglandin, such as alprostadil is formulated so as to not be mixed with a substantial amount, such as up to 0.5 weight %, or up to 0.4 weight % of a C₁-C₃ alcohol, such as ethanol.

In some embodiments, ethyl laurate can be partly or completely replaced by a fatty acid ester including without limitation isopropyl n-butyrate, isopropyl n-hexanoate, isopropyl n-decanoate, isopropyl myristate, isopropyl palmitate, octyldodecyl myristate, ethyl acetate, butyl acetate, methyl acetate, methylvalerate, methylpropionate, diethyl sebacate, ethyl oleate and mixtures thereof. The fatty acid ester is present in an amount of about 0.05-about 40% weight percent, or about 0.1-about 35% weight percent.

In some embodiments, prostaglandin E (PGE) group compounds are stabilized as non-aqueous compositions that are free of or substantially free from C₁-C₄ alcohols and include the prostaglandin E compound together with a (C₁-C₄)-alkyl(C₈-C₂₂)carboxylic ester (e.g., ethyl laurate). Preferably, an N,N-di(C₁-C₈)alkylamino substituted, (C₄-C₁₈)alkyl(C₂-C₁₈)carboxylic ester and/or a pharmaceutically acceptable addition salt thereof, and optionally, a viscosity enhancing agent such as a polysaccharide, a modified polysaccharide, a cellulose derivative, a cross-linked polyacrylic acid, or a salt thereof, can also be present in the composition.

In one embodiment, the composition is included in a packaged, multi-component dosage form, which comprises a sealed actives compartment containing a composition utilized in the invention, and a sealed diluent compartment containing a pharmaceutically compatible diluent for forming a prostaglandin E group compound-topical dosage form ready for administration. The diluent is combinable with the composition to provide another composition for topical application to a patient. The diluent can contain a C₁-C₄ alcohol (e.g., ethanol), water, and one or more buffering agents to provide a physiologically acceptable pH for the topical dosage form when the diluent is combined with the prostaglandin E group compound containing composition prior to use. The topical dosage form, which results from combination of the diluent and the prostaglandin E group compound containing composition preferably is in the form of a cream, a gel, or an ointment.

A preferred N,N-di(C₁-C₈)alkylamino substituted, (C₄-C₁₈)alkyl (C₂-C₁₈)carboxylic ester is dodecyl 2-(N,N-dimethylamino)-propionate, a pharmaceutically acceptable addition salt thereof, or a combination thereof.

In one preferred embodiment, the prostaglandin E composition is free of or substantially free from C₁-C₄ alcohols and comprises about 0.025 to 10 percent by weight of a prostaglandin E group compound, about 0.025 to 40 percent by weight of a N,N-di(C₁-C₈)alkylamino substituted, (C₄-C₁₈)alkyl(C₂-C₁₈)carboxylic ester skin permeation enhancer, such as dodecyl 2-(N,N-dimethylamino)-propionate or a salt thereof, about 0.25 to about 40 percent by weight of a (C₁-C₄)-alkyl(C₈-C₂₂)carboxylic ester (preferably ethyl laurate). Optionally, the composition can include about 0.05 to 40 percent by weight of a viscosity enhancing agent (e.g., guar gum).

A particularly preferred multi-part dosage form embodiment comprises a sealed actives compartment containing a PGE containing composition utilized in the invention, and a sealed diluent compartment containing a diluent for admixture with the PGE composition to provide a topical dosage form. The diluent comprises about 5 to 95 parts by weight of a C₁-C₄ alcohol (preferably ethanol), and about 5 to 95 parts by weight water, preferably an amount of one or more buffering agents sufficient to maintain a physiologically acceptable pH in a topical PGE dosage form, and optionally, about 0.001 to 5 parts by weight of an antifoam agent, in a sealed compartment of container. The sealed diluent compartment is packaged together with the sealed actives compartment, preferably in the form of a two-compartment pouch or packet, in which the two compartments are separated by a frangible seal. In some embodiments, instructional indicia, such as a label, pamphlet, booklet, brochure, video tape, CD-ROM, and the like can be included with the dosage form so as to inform the user about the contents, how to combine the diluent and prostaglandin E composition into a topical dosage form, how to apply the dosage form, and the like. The topical dosage form prepared by combining the diluent and prostaglandin E compound containing composition is viscous and substantially non-flowing, such as a cream, gel, or ointment.

Other compositions useful according to this invention are provided in or can be adapted from those disclosed in U.S. Pat. No. 7,560,489, which is incorporated herein in its entirety by reference.

The composition utilized herein can include a buffer system. Buffer systems serve to maintain or buffer the pH of compositions within a desired range. The term “buffer system” or “buffer” as used herein has reference to a solute agent or agents which, when in a water solution, stabilize such solution against a major change in pH (or hydrogen ion concentration or activity) when acids or bases are added thereto. Solute agent or agents which are thus responsible for a resistance to change in pH from a starting buffered pH value in the range indicated above are well known. While there are numerous other suitable buffers, such as acetate buffers, potassium phosphate monohydrate is employed in one embodiment of this invention.

The final pH value of the pharmaceutical composition of the present invention may vary within the physiologically compatible range. Necessarily, the final pH value is not irritating to human skin. Without violating this constraint, the pH may be selected to improve prostaglandin E₁ or another such agent's, stability and to adjust consistency when required. With these factors accounted for, the preferred pH value is about 3.0 to 7.4. The most preferred pH range is from about 3.5 to about 6.0.

Another component of the final combined composition utilized herein is water.

In one embodiment, the female patient is pre-menopausal. In another embodiment, the female patient is peri-menopausal. In another embodiment, the female patient is post-menopausal.

### EXAMPLE

### Example 1

### A Randomized, Single Center, Single-Blind, Crossover Thermographic Study to Evaluate the Effect of Femprox® (0.42% Alprostadil) Cream Compared to an Over-the-Counter Marketed Lubricant in the Absence of Sexual Stimulus in Healthy Women

An objective of this study was to demonstrate the effect on external genitalia of Femprox® (0.42% w/w Alprostadil) cream. Each unit dose of Femprox® cream includes 1000 micrograms (mcg) of Alprostadil in 238 mg cream. Femprox® was compared to an over-the-counter (OTC) marketed lubricant in the absence of sexual stimulus in healthy women using thermography. Another object was to demonstrate the subjective responses to the study medication using self-report questionnaires. Yet another objective was to demonstrate the safety and tolerability of topical Alprostadil cream applied to the genitals.

### Study Medication

The study medication is provided in a unit dose dispenser delivering 238 mg of cream containing 1000 μg of alprostadil as the active drug substance manufactured by Therapex Inc. (Quebec, Canada). In addition to alprostadil, the drug product includes the following: purified water, anhydrous ethanol, ethyl laurate, hydroxypropyl guar gum, dodecyl-2-N,N-dimethylaminopropionate hydrochloride (DDAIP), potassium dihydrogen phosphate, sodium hydroxide, phosphoric acid.

The control medication is ProAdvantage water soluble lubricating jelly in individual 3 gm sterile packets.

### Package, Code and Storage

Each dose of alprostadil is pre-measured and packaged in a unit dose dispenser. All study medication is packed as 1 dispenser/dose and contains 238 mg cream or approximately 250 microliters. The lubricant is drawn up in a single dose sterile syringe to equal approximately 250 microliters volume of lubricant.

### Design

A Phase I, single-blind, crossover study was conducted in ten (10) healthy, pre-menopausal women aged 18-49.

The study included two topical administrations for treatment within a two-week period. Subjects were randomized to receive a) topical Alprostadil cream 1000 mcg, or b) topical over-the-counter lubricant, at one visit, and cross over to the other study medication at their last visit.

### First Application

The subject is randomized to receive topical Alprostadil cream 1000 mcg, or topical over-the-counter lubricant. Before being placed into position for thermography the subject completes the Likert-style subjective questionnaire. The thermography camera is set up in the correct position for the particular subject. Subjects are required to sit for approximately 20-30 minutes so that baseline genital temperatures can be established. The room temperature is also recorded during this period. Study medication is then be applied by an unblinded health care provider (not the person performing thermography) who does not perform procedures other than study medication application at visit 2 or 3.

Study medication is applied directly to the subject's clitoris via the index finder followed by downward motion to the anterior vaginal wall, and the study medication is applied both to the clitoris, and peri-urethral anterior vaginal wall tissue.

After application, the thermography camera is adjusted. The subject maintain the lithotomy or frog leg position for the duration of the treatment period. To minimize artifact, subjects remain as still as possible during the testing procedure (approximately 1.5 hours). The thermal imaging camera are set up to record genital and thigh temperature for the duration of the treatment period focused on the labia majora. Camera distance is recorded and monitored. Thermography recording will begin at baseline and continue for 1 hour post application.

At the end of the treatment period, the subject washes the genital area with warm water and dry the area thoroughly. The subject is queried if any adverse event occurred during the treatment period or Protocol in between treatment period days.

Subjective responses to the study medication is assessed again, approximately 1 hour post dosing, by completing the post-dosing questionnaire.

### Second Application

After a minimum of 24 hours the subject returns for a second application of the crossover study medication. The application and temperature measurements and other functions are performed as for the first application.

### Results

TABLE 1
Treatment Comparison for Change from Baseline
in Temperature (All Subjects)

| | Least Squares Mean | | |
|---|---|---|---|
| Variable | Placebo | Femprox | P-value |
| Vestibule: | | | |
| Baseline | 34.52 | 34.70 | 0.6672 |
| Change to post-dose average | −1.04 | 0.56 | <0.0001 |
| Maximum post-dose temperature | 34.00 | 36.32 | <0.0001 |
| Clitoral | | | |
| Baseline | 34.24 | 34.52 | 0.4447 |
| Change to post-dose average | −0.85 | 0.51 | <0.0001 |
| Maximum post-dose temperature | 33.85 | 36.16 | <0.0001 |
| Vulvar | | | |
| Baseline | 33.98 | 34.22 | 0.5413 |
| Change to post-dose average | −0.84 | 0.26 | <0.0001 |
| Maximum post-dose temperature | 33.57 | 35.17 | 0.0014 |

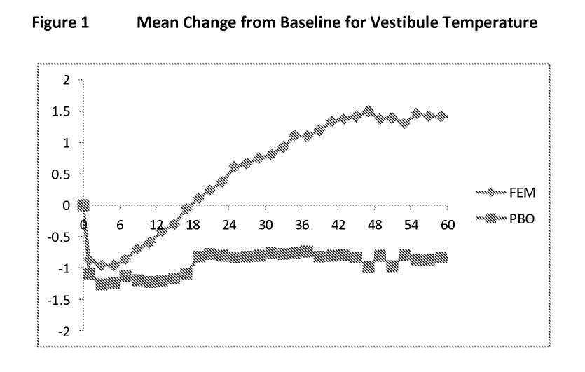

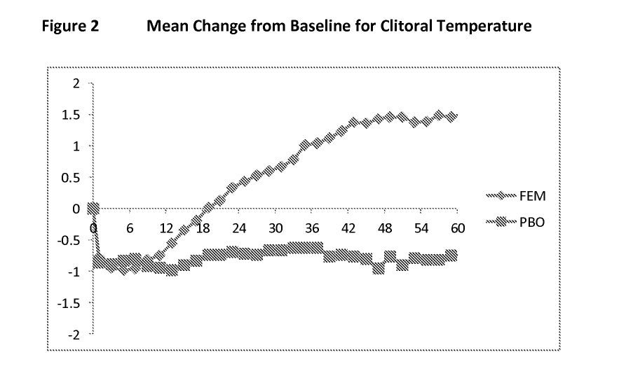

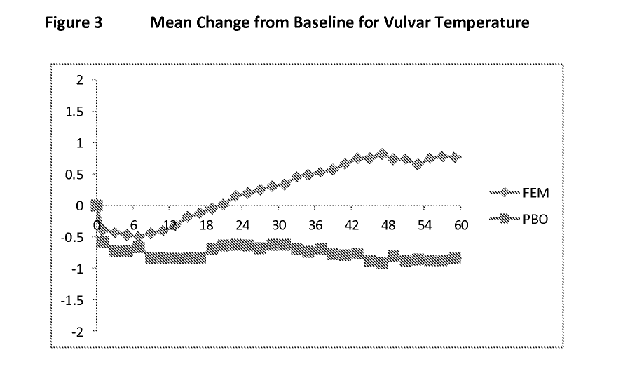

See also, FIGS. 1, 2, and 3.

As per these studies, sustained treatment differences began at 11 minutes postdose for vestibule, 19 minutes for clitoral, and 9 minutes for vulvar.

### Subjective Questionnaires

Using the scale below, subjects were asked to rate their current genital sensations, e.g., warmth in genitals, genital wetness or lubrication, tingling or fullness, in a scale of 1-5:

| 1 | 2 | 3 | 4 | 5 |
|---|---|---|---|---|
| Barely | | Neutral | | Intense |
| noticeable | | | | |

The responses are tabulated below.

| Question | Femprox | Placebo |
|---|---|---|
| Response | N = 9 | N = 3 |
| Intensity | | 0 |
| 1 - Barely noticeable | 0 | 0 |
| 2 | 0 | 0 |
| 3 - Neutral | 4 | 3 |
| 4 | 4 | 0 |
| 5 - Intense | 1 | 0 |
| Sensation description | | |
| 1 - Negative | 1 | 0 |
| 2 | 0 | 0 |
| 3 - Neutral | 2 | 3 |
| 4 | 6 | 0 |
| 5 - Positive | 0 | 0 |
| When most intense | | |
| 1 - Almost immediately | 3 | 1 |
| 2 - Within 5 to 10 minutes | 3 | 0 |
| 3 - More than 30 minutes | 3 | 1 |
| 4 - Could not tell | 0 | 1 |
| Duration of sensation | | |
| 1 - Less than 5 minutes | 0 | 0 |
| 2 - From 5 to 10 minutes | 2 | 1 |
| 3 = Longer than 30 minutes | 5 | 1 |
| 4 = Could not tell | 2 | 1 |

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

1. A method of increasing vestibule temperature in a female patient in need thereof, the method comprising topically administering a composition comprising an effective amount of alprostadil and an effective amount of dodecyl 2-(N,N-dimethylamino)propionate (DDAIP) and associated metabolites to the patient's genitalia.

2. A method of increasing clitoral temperature in a female patient in need thereof, the method comprising topically administering a composition comprising an effective amount of alprostadil and an effective amount of dodecyl 2-(N,N-dimethylamino)propionate (DDAIP) to the patient's genitalia.

3. A method of increasing vulvar temperature in a female patient in need thereof, the method comprising topically administering a composition comprising an effective amount of alprostadil and an effective amount of dodecyl 2-(N,N-dimethylamino)propionate (DDAIP) to the patient's genitalia.

4. A method of enhancing female sexual response during a sexual engagement, comprising topically administering a composition comprising an effective amount of alprostadil and an effective amount of dodecyl 2-(N,N-dimethylamino)propionate (DDAIP) to the genitalia of a female in need thereof between about 10 minutes to 90 minutes prior to the sexual engagement by the female, wherein one or more of vestibular, clitoral or vulvar temperature of the female is increased.

5. A method of enhancing female sexual response during a sexual engagement, comprising identifying a female in need thereof during a 120 minute window of time and topically administering a composition comprising an effective amount of alprostadil and an effective amount of dodecyl 2-(N,N-dimethylamino)propionate (DDAIP) to one or more of vestibule, clitoral or vulvar region of the female's genitalia between about 10 minutes prior to and up until about 100 minutes into said 120 minute window.

6. The method of claim 1, wherein an effective temperature increase is observed within about 5-about 20, or about 10 minutes.

7. The method of claim 6 wherein the effective temperature is maintained for about 45 minutes to about 2 hours, or about 1-about 1.5 hours.

8. The method of claim 1, wherein the composition is a cream.

9. The method of claim 1, wherein the composition comprises 0.42% weight percent of alprostadil.

10. The method of claim 1, wherein the composition comprises about 0.5 mg-2 mg, or about 1-1.5 mg, or 1 mg of alprostadil.

11. The method of claim 1, wherein the composition comprises 0.1-5% weight percent of DDAIP.

12. A method of enhancing female sexual response during a sexual engagement, comprising topically administering a composition to the patient's genitalia wherein said composition is configured to increase the temperature of one or more of the vestibule, clitoris and/or vulva.

13. The method of claim 12, wherein the composition comprises an effective amount of alprostadil and an effective amount of dodecyl 2-(N,N-dimethylamino)propionate (DDAIP) and associated metabolites.

14-18. (canceled)

19. The method of claim 1 wherein the vestibule temperature increase is at least 0.5% for a 90 minute duration.

20. The method of claim 3, wherein the vulvar temperature increase is at least 0.7% for a 90 minute duration.

21. The method of claim 2, wherein the clitoral temperature increase is at least 0.8% for a 90 minute duration.

22. The method of claim 2, wherein an effective temperature increase is observed within about 5-about 20, or about 10 minutes.

23. The method of claim 3, wherein an effective temperature increased is observed within about 5-about 20, or about 10 minutes.

24. The method of claim 2, wherein the composition is a cream.

25. The method of claim 3, wherein the composition is a cream.
